(19) United States
(12) Patent Application Publication
(10) Pub. No.: US 2010/0203575 A1
(43) Pub. Date: Aug. 12, 2010

(54) In Vitro Beating Heart Model

(75) Inventors: Marc Peschanski, Creteil (FR); Walter Habeler, Paris (FR); Christelle Monville, Champigny Sur Marne (FR)

Correspondence Address: MCANDREWS HELD & MALLOY, LTD, 500 WEST MADISON STREET, SUITE 3400, CHICAGO, IL, 60661, US

(73) Assignee: INSERM (Institut National de la Sante et de la Recherche Medicale), Paris (FR)

(21) Appl. No.: 12/677,202

(22) PCT Filed: Sep. 10, 2008
(86) PCT No.: PCT/EP2008/062005

§ 371 Date: Mar. 9, 2010

(30) Foreign Application Priority Data
Sep. 11, 2007 (EP) 07301357.5

(51) Int. Cl. C12Q 1/02; C12N 5/071; C12N 5/073

(52) U.S. Cl. 435/29; 435/395

(57) ABSTRACT

The invention relates to a method for preparing a heart organotypic slice culture, comprising; a) providing a slice of heart, b) placing the slice of step a) on the upper surface of a semiporous support which is permeable to a culture medium, c) placing the support of step b) onto a culture medium, the slice being fed through the semi-porous support by capillarity.

### FIELD OF THE INVENTION

The present invention relates to a method for culturing heart slices.

### BACKGROUND OF THE INVENTION

Cardiovascular diseases are the leading cause of death in Europe and North America (Ott et al., 2006). Simple ex vivo or in vitro models are most useful for testing putative therapy protocols, as they allow quick and controlled screening of variants and possible improvements (Sanchez et al 2006). Heart function critically depends on an intact structure on the tissue level, characterized by cardiomyocyte size and orientation, gap junction distribution and properties of non-myogenic cells as well as extracellular matrix (Halbach et al. 2006). To date, relatively little progress has been made in establishing a viable heart slice preparation (Parrish et al. 1995; Yamashita et al. 2004). This is in part due to the fact that the heart contains a significant amount of extracellular matrix and is expected to exhibit hypercontraction in response to even small increases in extracellular K₊, which leaks out of damaged cells (Pillekamp et al. 2005). Recently, Pillekamp and colleagues (2006) reported the establishment of an ischemic heart slice model and the slices were cultured only for a short time.

A three-dimensional model could be very useful for drug safety testing in many cardiac diseases like Long QT syndrome type 1, an autosomal disorder with syncope or sudden death due to ventricular tachyarrhythmias caused by mutations of the potassium channel gene KCNQ1 (Zareba et al. 2003) where screening for drug-induced QT-interval prolongation is important (Shah et al. 2004).

Several experimental and clinic observations suggest that cell transplantation could be beneficial to restore the cardiac function. Human embryonic stem (hES) cells can divide indefinitely and give rise to many cellular types, including cardiomyocytes. hES cells represent a very promising tool for a cell approach in heart failures (Laflamme et al., Tomescot et al., 2007). Several reports suggest that embryonic stem cells represent a very important tool in cell therapy approach for cardiovascular repair (Gerecht-Nir et al.2003, Menasché 2004; Hodgson et al 2004). Indeed, several studies have shown that transplantation of ES cells into injured heart generated functional cardiomyocytes and improve cardiac functions. However, several aspects like migration and cell survival are limiting factor for cell therapy approach.

Existing models for heart function include measurements of single cells (Metzger et al., 1994; Dolnikov et al., 2005 (or in an artificial extracellular matrix (Davis et al., 2005; Baharvand et al., 2005). However, those systems do not reflect the clinical situation. Moreover, recent studies indicate that embryonic tissue has unique properties influencing survival and differentiation (Eisenberg et al., 2006).

Thus, there is an unmet need for providing a heart slice culture model which enables a long-term culture in order to study heart physiology, to screen drugs and to study cell transplantation.

### SUMMARY OF THE INVENTION

The present invention relates to a method for preparing a heart organotypic slice culture, comprising:
a) providing a slice of heart,
b) placing the slice of step a) on the upper surface of a semiporous support which is permeable to a culture medium,
c) placing the support of step b) onto a culture medium, the slice being fed through the semi-porous support by capillarity.

The invention also relates to a system comprising a heart organotypic slice culture on a semiporous support.

The invention also relates to the use of a system according to the invention for studying heart physiology, for screening drugs or for studying cell transplantation.

### DETAILED DESCRIPTION OF THE INVENTION

The present invention relates to a method for preparing a heart organotypic slice culture, comprising:
a) providing a slice of heart,
b) placing the slice of step a) on the upper surface of a semiporous support which is permeable to a culture medium,
c) placing the support of step b) onto a culture medium, the slice being fed through the semi-porous support by capillarity.

As used herein, the term “heart organotypic slice” refers to a slice of heart which is obtainable from an isolated mammalian heart and retains the three-dimensional connectivity of the intact organ. The cell-cell interactions are preserved, and there is no selection of a particular cell type among the different cell types that constitute the organ. Several methods for obtaining heart organotypic slices are known to the skilled person and described in the art. Those include, for example, slicing using a vibratome, agarose embedding followed by sectioning by a microtome, or slicing using a heart matrix.

Typically, the heart organotypic slice according to the invention is obtainable from a mammalian heart, preferably, a rodent heart or a primate heart.

The heart organotypic slice according to the invention can be obtained from embryonic mammals, neonatal mammals or adult mammals. In a preferred embodiment, the heart organotypic slice of the invention is obtained from embryonic mammals. It may be obtained from the heart ventricles.

Typically, the thickness of the heart organotypic slice according to the invention is comprised between 300 and 1200 μm, preferably between 800 and 1200 μm. In a preferred embodiment, the thickness of the heart organotypic slice is about 1000 μm.

As used herein, the term “semiporous support” refers to a support which is permeable to certain molecules and impermeable to others. Typically, the semi-porous support according to the invention is permeable to a culture medium and enables the passage of nutrients and metabolic waste to and from the slice respectively.

In a preferred embodiment, the semiporous support comprises a semiporous membrane sealed to a polystyrene holder, such as the culture plate inserts manufactured as Millicell® (by Millipore, France). Those inserts are available either as hanging inserts, standing inserts or standing inserts with a low height which enable to fit the inserts inside a Petri dish.

The membrane can be made out of a variety of materials which are suitable for cell culture, such as hydrophilic polytetrafluoroethylene (PTFE, also known as Teflon™), mixed cellulose esters, polycarbonate, polyethylene terephthalate, or inorganic aluminium oxide (also known as Anopore™).

In one embodiment, the membrane of the semiporous support is coated with molecules which provide an appropriate extracellular environment for the heart organotypic slice, such as cell attachment molecules and extracellular matrix components. Typically, such molecules can include poly-ornithine collagens or proteoglycans.

Procedures for coating semiporous membranes are standard coating procedures known to those skilled in the art. Typically, one covers the membrane to be coated with an aqueous solution containing the coating molecule at the desired concentration. After an incubation period, the solution is removed, and the membrane is washed in order to remove any excess material.

A culture medium suitable for the method of the invention is any medium which provides the appropriate physicochemical environment to the slice. Suitable media are well known in the art and are commercially available from a variety of manufacturers, such as Gibco (Invitogen, France). It contains essential nutrients and can be supplemented with various growth factors, serum, antibiotics etc.

Typically, in the method according to the invention, the culture medium is replaced, partially or totally, at regular intervals, for example every 2 days, or every 3 days. In the method according to the invention, the slices are cultured in standard cell or tissue culture conditions. For example, the slices can be placed in an incubator, which provides an atmosphere containing 5% CO₂, and which maintains a temperature of about 37° C.

The method according to the invention enables the long-term in vitro culture of beating heart slices. As used herein, the term “long-term” refers to cultures which are viable for more than 2 weeks, preferably, more than 3 weeks, even more preferably more than 4 weeks. Typically, the cultures according to the invention are viable for more than 2 months, preferably more than 3 months. The viability of the slice can be assessed by a variety of methods well known in the art, such as the ability of the slice to beat, i.e. to exhibit spontaneous contractions and the cardiac structural architecture by immunohistochemistry.

The invention also relates to a system comprising a heart organotypic slice culture on a semi-porous support.

The invention also relates to the use of a system comprising a heart organotypic slice culture on a semi-porous support for studying heart physiology, for screening drugs or for studying cell transplantation.

The invention also relates to a method for studying heart physiology comprising the steps of:
a) providing a slice of heart,
b) placing the slice of step a) on the upper surface of a semiporous support which is permeable to a culture medium,
c) placing the support of step b) onto a culture medium, the slice being fed through the semi-porous support by capillarity,
d) studying the contractions in said slice.

As used herein, the expression “studying heart physiology” refers to the study of the function of this organ, in normal or pathological conditions. These studies include, but are not limited to, studies of hearts obtained from healthy animals or from animal models of a disease. The studies can include the study of spontaneous contractions, contractions in response to a variety of physiological and/or pathological stimuli or in response to various drugs such as epinephrine.

The invention also relates to a method for screening drugs which comprises the steps of:
a) providing a slice of heart,
b) placing the slice of step a) on the upper surface of a semiporous support which is permeable to a culture medium,
c) placing the support of step b) onto a culture medium, the slice being fed through the semi-porous support by capillarity,
d) adding a drug to the culture medium,
e) studying the contractions in said slice.

As used herein, the expression “screening drugs” includes screening drugs for modulating cardiac function, screening drugs for cardiac toxicity etc. This can be performed both on healthy and on diseased heart slices.

The invention also relates to a method for studying cell transplantation comprising the steps of:
a) providing a slice of heart,
b) placing the slice of step a) on the upper surface of a semiporous support which is permeable to a culture medium,
c) placing the support of step b) onto a culture medium, the slice being fed through the semi-porous support by capillarity,
d) transplanting cells into said slice.

As used herein the expression “studying cell transplantation” refers to transplantation of cells into the heart slices according to the invention. Such a use of system according to the invention is useful for cell therapy studies. Typically, cell transplantation according to the invention encompasses ES cell transplantation within the slice and the subsequent analysis of the migration and/or differentiation of said ES cells within the slice. According to one embodiment, the ES cells can be genetically modified to express molecules for improving cardiac function. According to one embodiment, the ES cells can be associated with a biocompatible porous scaffold in order to improve their transplantation into the slice. In a preferred embodiment, said scaffold is biodegradable, i.e., degrades over time within the slice and is eventually replaced entirely by cells or extracellular matrix. In a preferred embodiment, said biocompatible scaffold also includes biomolecules such as growth factors.

The invention will further be illustrated in view of the figures and examples.

### FIGURE LEGENDS

FIG. 1. Schematic representation of preparation of organotypic heart slices. (A) Ventricles of neonatal rat were placed on heart matrix and cut at a thickness of 1 mm. (B) The slices were placed on Millipore membrane and cultures at 37° C. in 4%CO₂ humidified atmosphere for up two month. (C) Stereomicroscope view of heart slice culture. Histological analysis of heart slices cultured for 1 month showed a well preserved cardiac architecture: (D) Hematoxylin-eosine, (E) anti-troponin I and (F) anti-CD31 staining. The nucleus were stained with DAPI (in blue).

FIG. 2. Time-change of spontaneous beating frequencies.

(A) Monitoring of spontaneous beating of heart slice cultures of four representative heart slice cultures. (B) Ca2+ spiking within selected ROI of heart slice loaded with fluo-4. The records are expressed as ΔF/F0, where F0 is the lowest fluorescent recorded. (C) Immunofluorescence of crysection showed uniform distribution of connexin 43 (in green) between rat cardiomyocytes of heart slice culture. The nuclei are stained blue with DAPI.

FIG. 3. Effect of epinephrine on contraction of heart slice cultures. (A) Epinepfrine increased the frequencies of spontaneous beating in dose-response manner. n=23, *p≦350.01 and **p≦0.001 compared with control (B) Monitoring of Ca230 spiking of untreated or treated with 1 μmol/L epinephrine of heart slice culture loaded with fluo-4. (C) Epinephrine treatment increases the frequencies of beating in human heart slices.

FIG. 4. Cell transplantation in heart slice cultures. (A) GFP-hES positive cells were injected into heart slices. (B) Epifluorescence observation, using stereomicroscope, showed the presence of GFP positive cells in the cardiac parenchyma close to injection site. (C) Observation of spontaneous beating frequency of heart slice injected with human embryonic stem cell (hES), mouse skeletal myoblastic cell (C2C12) and human embryonic kidney (HEK-293T) cells.

FIG. 5. Engraftment of undifferentiated human embryonic stem (hES) cells into organotypic heart slices. Heart slices were injected with 1×10⁴ hES cells were injected into heart slice. Presence of human cells, into the cardiac parenchyma, 60 days after injection. Human nuclei (red), Troponin I (green) and DAPI (blue) staining. Magnification ×10.

FIG. 6. Expression of human cardiac markers. Detection of hES cell-derived cardiomyocites into cardiac parenchyma using immunofluorescence analysis for human cardiac markers. (A) anti-human actinin, (B) anti-human desmin, and (C) anti-human Ki67 staining. Magnification ×40 (A-B) and X20 (C).

(D-E) Real time PCR analysis for human atrial nautrietic peptide (ANP) and human myosin light chain 2a (MLC2a) expression in heart slices two month after the injection of undifferentiated human ES cells (#1, #2 and #3). (F) rat UbC was used as internal control. The figures show both the profile of the melting curves of amplicons and the amplicons on gel.

### EXAMPLE

### Material and Methods:

Experimentation was performed in strict accordance with the recommendations of the European Ethical Committee (EEC) (86/609 EEC), and French National Ethical Committee (87/848) for care and use of laboratory animals. The human foetuses were obtained from abortion with the parental consent.

Organotypic Ventricular Slice Cultures, Set Up and Characterization

Cultures were prepared from ventricular sections of 3 day-old rats (Charles River, France) or 8 weeks-old human embyros. The hearts were removed and placed in Phosphate-Buffered Saline (PBS), atria were removed and the ventricles sliced at 1 mm-thickness using a rodent heart matrix (Harvard Apparatus). The heart slices were immediately transferred to a Millicell-CM 0.4 μm membrane (Millipore, France) and the insert placed into a 6 well-plate containing 1 ml of medium.

The culture medium consisted of DMEM/F12 medium supplemented with 20% Knockout Serum Replacement, 1% non essential amino acids, 2 mM L-glutamine, 0.1 mM β-mercaptoethanol, and 1% PS (Invitrogen, France). Heart slices were maintained for 30-80 days at 37° C. in a humidified atmosphere containing 5% CO₂.

Medium was changed three times a week.

Slices were observed under an inverted microscope every two days and their beating characterized by the observer in terms of frequency and localization of the most apparent contractions.

Further functional characterization of the contractile function of ventricular slices was carried out on 24 slices, at 30 days after plating, using a stereomicroscope and a temperature-controlled stage set at 37° C., following incubation with 10, 100nM and 1 μM of epinephrine (Sigma, France). Beating frequency was monitored 3 min after pharmacological stimulation.

Additional analysis was performed on 3 slices, at 30 days after plating using a confocal laser scanning microscope (Zeiss), following incubation with 5 μM fluo-4 AM (Invitrogen, France) for 20 min at 37° C.

Intra-Slice Implantation of Human Embryonic Stem Cells

In order to test whether the beating observed was due to the transplanted cells or was slice specific we injected different cell types. 293T human kidney (HEK-293T) and mouse skeletal myoblastic (C2C12) cell lines were obtained from American Type Culture Collection and grown in DMEM supplemented with 10% FCS and 1% L-glutamine. Human embryonic stem cells (SA01, cellartis, Sweden) were maintained on inactivated mouse embryonic fibrobrast feeder cells (STO, ATCC) in Dulbecco's modified Eagle's medium/F12 supplemented with 20% Knockout serum replacement, 1% non essential amino acids, 2mM L-glutamine, 0.1 mM β-mercaptoethanol, and 4 ng/ml basic fibroblast growth factor (bFGF) (Invitrogen, France), Undifferentiated hES, 293T and C2C12 cells were harvested and resuspended in the medium used for the culturing the organotypic slices at a density of 1×10⁵ cells/μl. 0.1 μl of this cell suspension were then injected, using 0, 5-μl Hamilton syringe fixed to the arm of a micromanipulator, into heart slice cultures. The contraction rate of heart slices was monitored by daily observation using Zeiss-Lumar V12 stereomicroscope.

In some experiments, the migration of transplanted hES cells into the ventricular slice tissue was evaluated. Then, undifferentiated human stem cells were incubated for 10 min with a 1:200 dilution of carboxyfluorescein succinimidyl ester (CFSE) at 37° C. in the dark. The cell suspension was washed with PBS supplemented with 5% FCS and resuspended in heart slice medium. CFSE-Ioaded hES cells were injected into ventricular slice cultures (1×10⁴ cells per slice) and monitored using the epifluorescence stereomicroscope every two days.

Gene expression. Total RNA was extracted from heart slice cultures using TRIZOL Reagent (Invitrogen, France) according to the manufacturer's protocol. RNA (1 μg) was reverse-transcribed using SuperScript II RNase H-Reverse Transcriptase (Invitrogen, France). cDNA was used as a template for gene expression analysis of human cardiac markers. The following primer pairs were used: human-MLC2a fwd: 5′-GAG-GAG-AAT-GGC-CAG-CAG-GAA-3′ (SEQ ID N^{o} 1) and rev: 5′-GCG-AAC-ATC-TGC-TCC-ACC-TCA-3′. (SEQ ID N^{o} 2). Murine β-tubulin amplification was performed as a control using primers fwd: 5′-CCG-GAC-AGT-GTG-GCA-ACC-AGA-TCGG-3′ (SEQ ID N^{o} 3) and rev: 5′-TGG-CCA-AAA-GGA-CCT-GAG-CGA-ACGG-3′. (SEQ ID N^{o} 4). PCR reaction was performed with an initial denaturation step at 94° C. for 5 min, followed by 35 cycles of 30 s at 94° C., 30 s at 55° C. and 30s at 72° C.

Protein content. At different times, ventricular slices were fixed for histological and immunological staining. Heart slices fixed in 4% paraformaldehyde for 2 h at 4° C. were cut into 10 μm thick sections. The cryosections were washed three times with PBS and then incubated for 1 h at room temperature with a saturating solution consisting of 5% normal goat serum, 5% normal horse serum in PBS-0.6% Triton X-100 (Sigma, France). Slides were incubated overnight at 4° C. with the following primary antibodies used at 1:400: anti-human nuclei mouse monoclonal (HNA) [Chemicon], anti-connexin 43 mouse monoclonal (CX43) [Chemicon], anti-rat CD31 mouse monoclonal [BD Pharmigen], anti troponin I rabbit polyclonal (cTnI) [Chemicon], anti-desmin mouse monoclonal [Chemicon], anti-human alpha-actinin mouse monoclonal [Chemicon]. After three washes with PBS, the slides were incubated with a 1:500 dilution of fluorescent-conjugated secondary antibodies for 1 h at room temperature. Secondary antibodies used were: Alexa-Fluor 488 goat anti-rabbit IgG and Alexa-Fluor 555 goat anti-mouse IgG [Molecular Probes, France]. After three washes the slices were incubated with 1/10000 DAPI and then the slides were mounted by using Fluoromount-G (Southern Biotech). The slides were observed under an epifluorescence microscope (Zeiss Imager Z1), and images processed using the Axio-vision software.

### Results:

Generation of Functional Ventricular Organotypic Slice Cultures

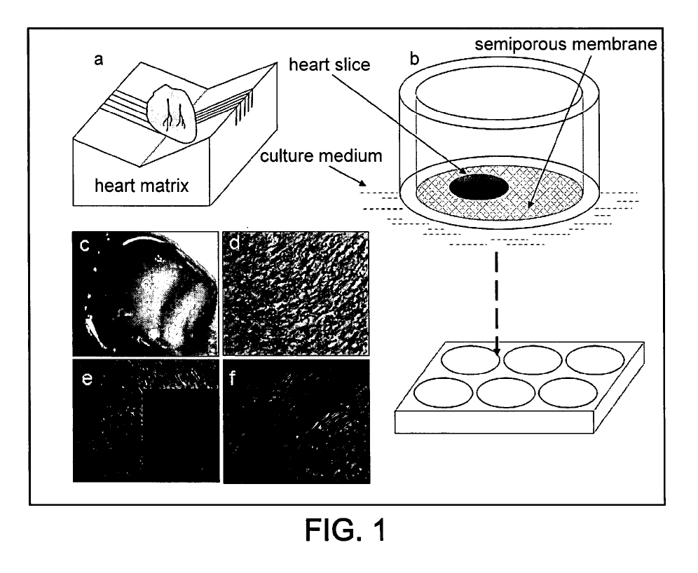

Organotypic heart slice cultures were prepared from heart of 3 days-old-rats. After removal of atria, the ventricles were placed on rodent heart matrix to slice them in sagittal section of 1 mm wide (FIG. 1A). The heart slices were placed onto semiporous millicell membranes inserts, placed in six well plates and cultured for 1 month in serum free condition (FIG. 1B). In this system the heart slices were fed by capillarity from the medium located underneath the insert. The FIG. 1C shows the stereomicroscopic aspect of a heart slice. H&E (FIG. 1 D) and troponinl staining (FIG. 1E) analysis showed well preserved cardiac architecture. Immunostaining with anti-CD31 antibody showed normal vessel structures in the cardiac tissue after long-term culture. (FIG. 1F).

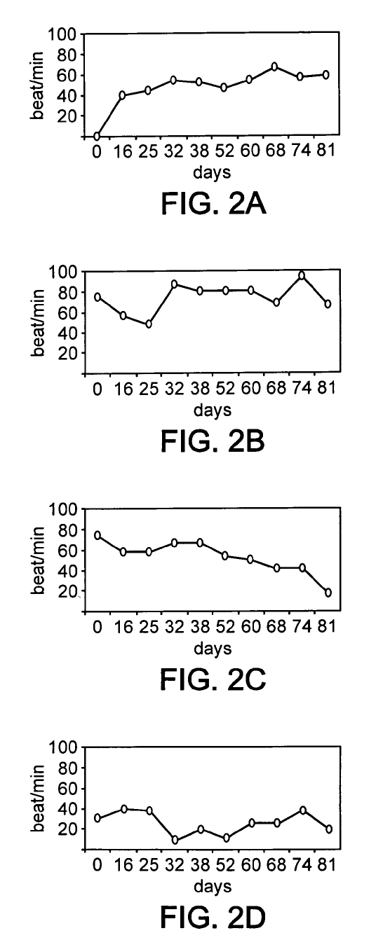

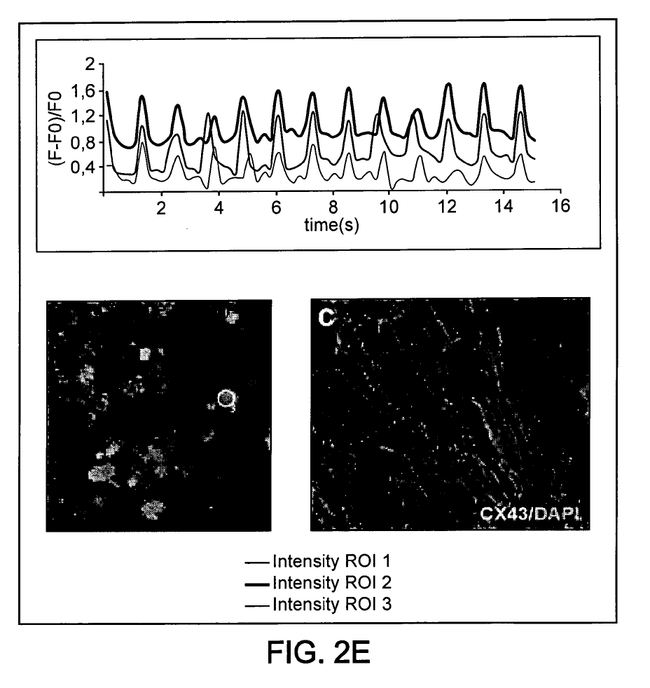

Stereomicroscope observation revealed the presence of spontaneous contraction in the majority of heart slices (data not shown). FIG. 2A shows the time change of spontaneous beating frequency of four representative heart slice monitored at 37° C. To investigate the influence of temperature on contraction spontaneous the heart slice cultures were monitored at different temperature (25° C.). Changes in temperature had not impact in the time course of contraction of heart slices (data not shown).

To monitor spontaneous Ca2+ spiking, heart slice cultures, were loaded with 5 μmol/L Fluo-4 AM ester and the temporal change of fluorescent intensity was measured at physiological temperature (37° C.). Confocal analysis showed rhythmic Ca2+ oscillation in different region of heart slice (video and FIG. 2B). As shown in FIG. 2C, immunofluorescence staining of heart slices indicates uniform distribution of connexin 43 between rat cardiomyocytes.

Similar long-term beating was observed using heart slices from an 8 week-old donor embryo (data not shown)

Pharmacological Response of Heart Slice to Beta-Adrenergic Stimulation

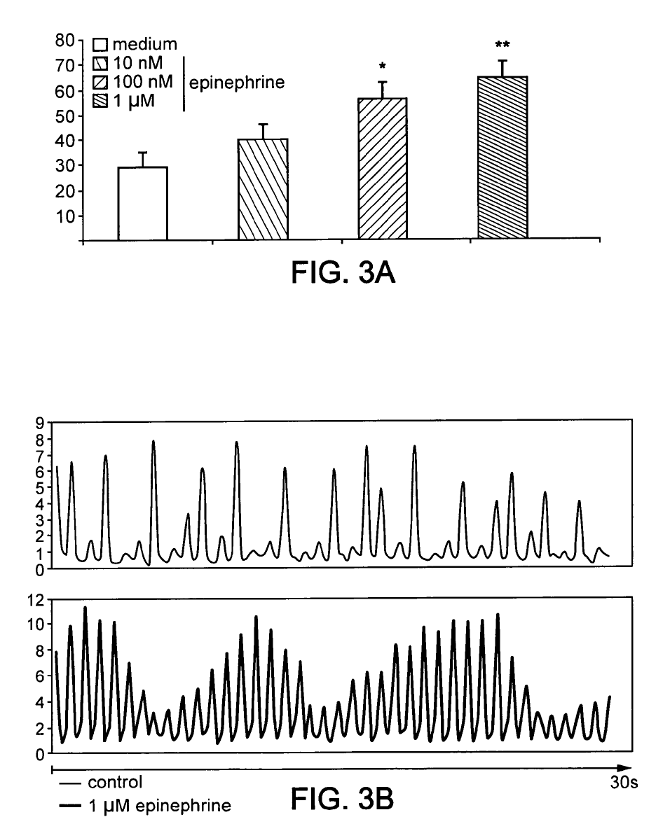

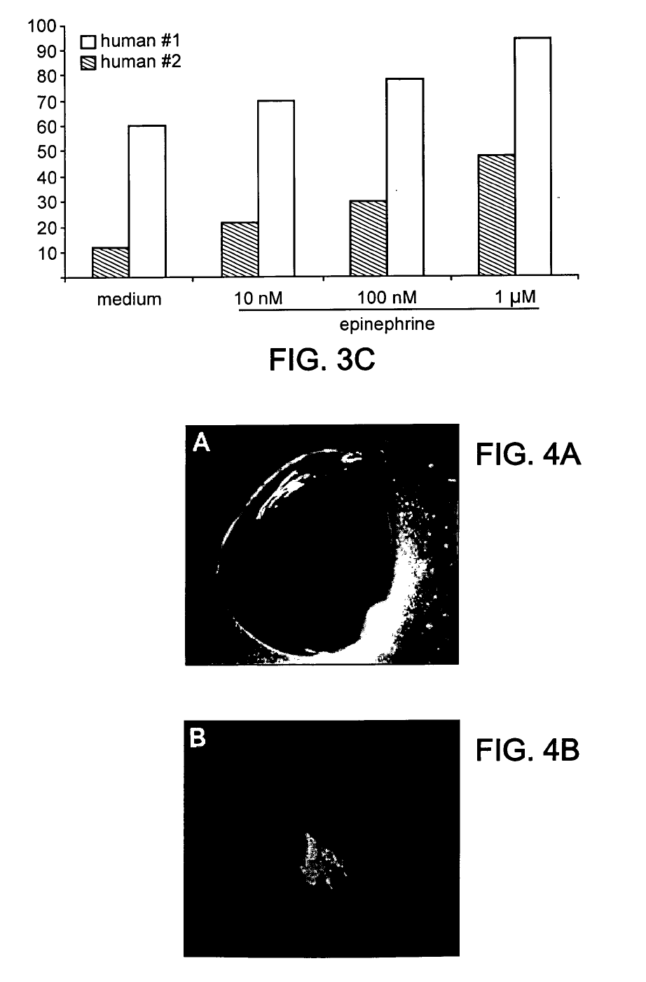

To evaluate the integrity of β-adrenergic response and in turn inotropic and chronotropic potentials of our models, heart slices were treated with 10, 100 nmol/L and 1 μmol/L of epinephrine. The treatment with epinephrine increased the spontaneous beating frequencies of heart slice in dose-response manner (FIG. 3A). Moreover, confocal analysis of calcium currents of heart slice, loaded with fluo-4, showed a dose-dependent increase in Ca2₊-transient amplitude and frequency when the heart slices were treated with epinephrine (FIG. 3B). Maximal response was obtained with 1 μmol/L epinephrine.

Similar findings were also obtained from human heart slices. As shows in FIG. 3C, addition of epinephrine increased spontaneous beating frequency of both human heart slices, in a dose-dependent manner.

Cell Transplantation

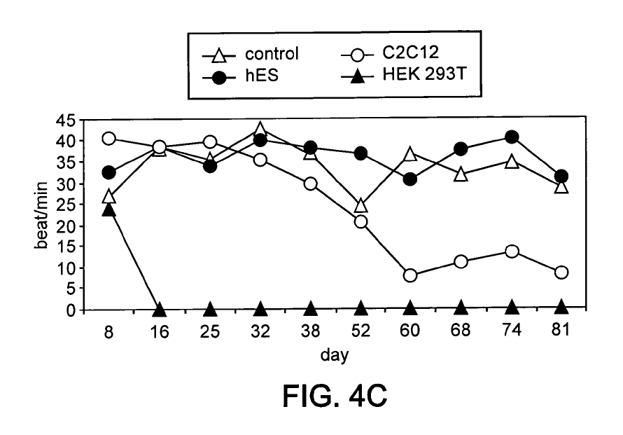

To address whether heart slice cultures could be used as an ex-vivo cell transplantation model, GFP-expressing hES cells were microinjected into heart slice cultures. Epifluorescence observation, using stereomicroscope, showed the presence of GFP positive cell in the cardiac parenchyma close to the injection site (FIG. 4A-B). In our model it is possible to monitor GFP positive cells using epifluorescence stereomicroscope without fixation of cardiac tissue and heart slices cultures were monitored daily to evaluate the effect of cell transplantation in the spontaneous contraction. Three different cell types were injected: (i) human embryonic stem cell (hES), (ii) human embryonic kidney cell (HEK-293T) and (iii) mouse skeletal myoblastic cell line (C2C12). FIG. 3C shows the time course of beating of heart slice observed at 37° C. Injection of HES and C2C12 cells had not effect on spontaneous contraction over the 81 days of observation. In sharp contrast, the inhibition in the beating frequency was observed after 11 days in the heart slice cultured injected with HEK-293T cells. Histological analysis showed that the inhibition of spontaneous contraction was due to high proliferation of HEK-293T cells that colonized the entire surface of slices probably causing hypoxia (data not shown).

Cardiomyocyte Differentiation of hES Cells in Heart Slices Culture

To evaluate the differentiation of hES cells into cardiomyocytes in our model, 1×10⁴ undifferentiated hES cells were injected into heart slices and the slices were cultured, for 60 days, in serum free condition.

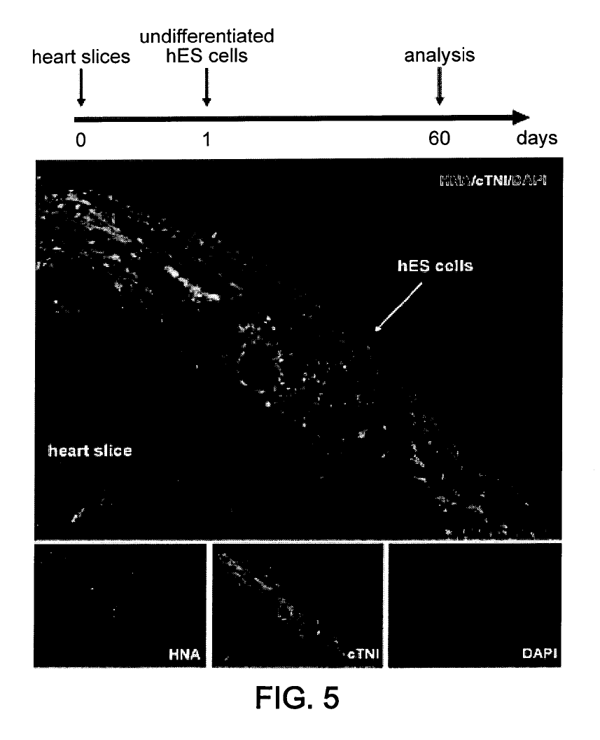

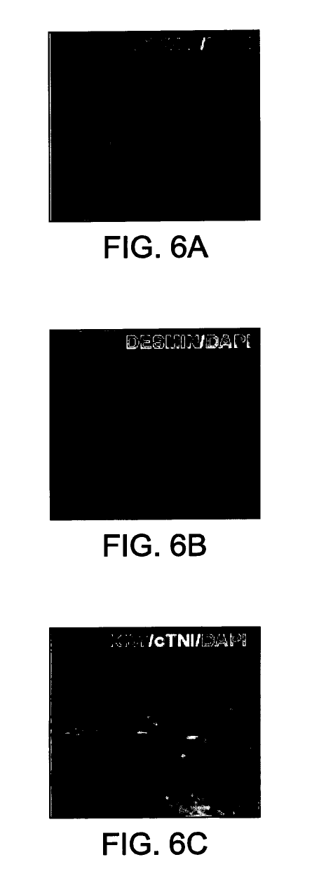

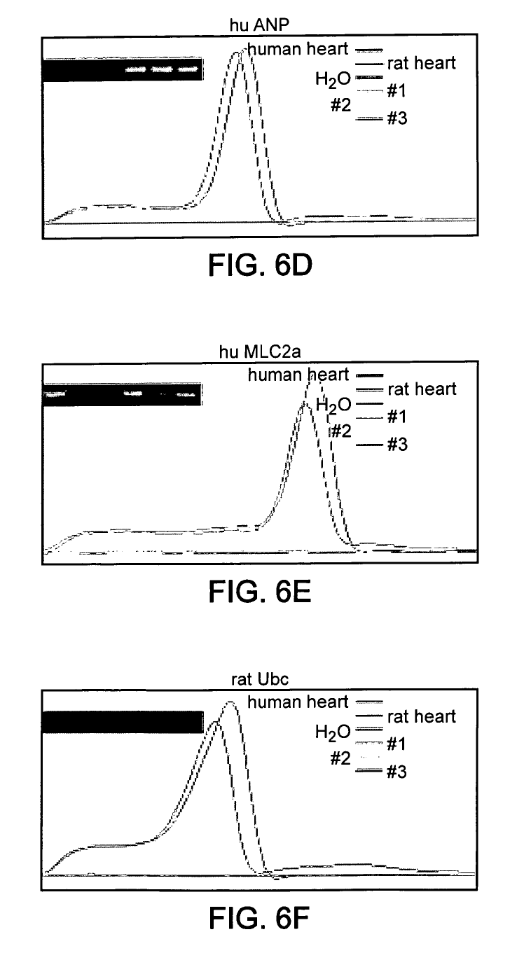

Immunofluorescence analysis showed the presence of human cells in the heart slice (FIG. 5). The human cells lost rounded morphology to show a spread morphology suggesting they started to differentiate when grafted into the cardiac tissue. The expression of several human cardiomyocyte markers was evaluated by immunostaining in heart slice cultures. As shown in FIG. 6, two months after injection, the hES-derived cardiomyocytes expressed alpha actinin (FIG. 6A) and desmin (FIG. 6B).

In addition, we have also used real-time PCR analysis to evaluate the expression of cardiac differentiation genes. Real-time PCR assay indicated that human atrial nautrietic peptide (ANP) and human myosin light chain 2a (MLC-2a), both markers of cardiac differentiation, were expressed in hES-derived cardiomyocytes injected in heart slice cultures (FIG. 6D-E).

Moreover, two months after injection, staining with an anti-Ki67 antibody, showed the presence of significant numbers of dividing hES cells (FIG. 6C).

Adult cardiomyocytes are not migratory cells; we investigated the migration of hES cell transplantated in the cardiac tissue of heart slices. Undifferentiated hES cells were loaded with CFSE and injected into heart slices. The transplantated cells were easily visualized under epifluorescence stereomicroscope shows a representative time course observation of a heart slice injected with CFSE-loaded hES cells, 3, 10 and 20 days after injection (or data not shown). Our observation showed that the hES cells were restricted to the area near the injection site.

Thus, the present inventors demonstrate for the first time that a long-term viable ventricular slice culture can be generated from 3-days-old rats' hearts and from 8 week-old human embryos used for pharmacological and cell therapy studies. The viability of this preparation was further validated by the fact that the slices exhibited spontaneous beating for up to 2 or even 3 months after preparation and that the excitation spread throughout the whole slice. While spontaneous beating in adult cardiomyocytes indicates cell damage and Ca²⁺ overload, it is a typical property of healthy embryonic heart cells (Fleischmann et al., 2004). Thus, the present invention discloses a method for providing an in vitro beating heart model.

### REFERENCES

Baharvand H, Azarnia M, Parivar K, Ashtiani S K. The effect of extracellular matrix on embryonic stem cell-derived cardiomyocytes. J Mol Cell Cardiol. 2005; 38: 495-503.

Davis M E, Hsieh P C, Grodzinsky A J, Lee R T. Custom design of the cardiac microenvironment with biomaterials. Circ Res. 2005 Jul. 8; 97(1):8-15. Review.

Dolnikov K, Shilkrut M, Zeevi-Levin N, Danon A, Gerecht-Nir S, ltskovitz-Eldor J, Binah O. Functional properties of human embryonic stem cell-derived cardiomyocytes. Ann NY Acad Sci. 2005; 1047: 66-67.

Eisenberg L M, Eisenberg C A. Embryonic myocardium shows increased longevity as a functional tissue when cultured in the presence of a noncardiac tissue layer. Tissue Eng. 2006; 12: 853-865.

Fleischmann B K, Duan Y, Fan Y, Schoneberg T, Ehlich A, Lenka N, Viatchenko-Karpinski S, Pott L, Hescheler J, Fakler B. Differential subunit composition of the G protein-activated inward-rectifier potassium channel during cardiac development. J Clin Invest. 2004 October; 114(7):994-1001.

Gerecht-Nir S, Ziskind A, Cohen S, Itskowitz-Eldor J. Human embryonic stem cells as an in vitro model for human vascular development and the induction of vascular differentiation. Lab Invest. 2003 December; 83(12):1811-20.

Halbach M, Pillekamp F, Brockmeier K, Hescheler J, Müller-Ehmsen J, Reppel M. Ventricular slices of adult mouse hearts—a new multicellular in vitro model for electrophysiological studies. Cell Physiol Biochem. 2006; 18:01-08.

Hodgson D M, Behfar A, Zingman L V, Kane G C, Perez-Terzic C, Alekseev A E, Puceat M, Terzic A. Stable benefit of embryonic stem cell therapy in myocardial infarction. Am J Physiol Heart Circ Physiol. 2004 August; 287(2):H471-9.

Laflamme M A, gold J, Xu C, Hassanipour M, Rosier E, Police S, Muskheli V, murry C E. Formation of human myocardium in the rat heart from human embryonic stem cells. Am J Pathol. 2005; 167: 663-671.

Menasché P. Embryonic stem cells pace the heart. Nat Biotechnol. 2004; 22: 1237-1238.

Metzger J M, Lin W I, Samuelson L C. Transition in cardiac contractile sensitivity to calcium during the in vitro differentiation of mouse embryonic stem cells. J Cell Biol. 1994 August; 126(3):701-11.

Ott H C, Taylor D A. From cardiac repair to cardiac regeneration-ready to translate? Expert Opin Biol Ther. 2006; 6: 867-78.

Parrish A R, Gandolfi A J, Brendel K. Precision-cut tissue slices: applications in pharmacology and toxicology. Life Sci. 1995; 57; 1887-1901.

Pillekamp F, Reppel M, Dinkelacker V, Duan Y, Jazmati N, Bloch W, Brockmeier K, Hescheler J, Fleischmann BK, Koehling R. Establishment and characterization of a mouse embryonic heart slice preparation. Cell Physiol Biochem. 2005; 16: 127-132.

Sanchez A, Fernandez M E, Rodriguez A, Fernandez J, Torre-Perez N, Hurlé J M, Garcia-Sancho J. Experimental models for cardiac regeneration. Nature Clinical Practice Cardiovascular Medicine 2006; 3: S29-S32.

Shah R R. Drug-induced QT interval prolongation: regulatory perspectives and drug development. Ann Med. 2004;36 Suppl 1:47-52.

Tomescot A, Leschik J, Bellamy V, Dubois G, Messas E, Bruneval P, Desnos M, Hagege A A, Amit M, Itskovitz J, Menasché P, Pucéat M. Differentiation in vivo of cardiac committed human embryonic stem cells in post-myocardial infracted rats. Stem Cells. 2007; 31.

Yamashita D, Kohzuki H, Kitagawa, Y, Nakashima T, Kikuta A, Takaki M. O2 consumption of mechanically unloaded contractions of mouse left ventricular myocardial slices. Am J Physiol Heart Circ Physiol 2004; 287; 54-62.

Zareba W, Moss A J, Sheu G, Kaufman E S, Priori S, Vincent G M, Towbin J A, Benhorin J, Schwartz P J, Napolitano C, Hall W J, Keating M T, Qi M, Robinson J L, Andrews M L; International LQTS Registry, University of Rochester, Rochester, N.Y. Location of mutation in the KCNQ1 and phenotypic presentation of long QT syndrome. J Cardiovasc Electrophysiol. 2003 November; 14(11):1149-53.

1. A method for preparing a heart organotypic slice culture, comprising:
a) providing a slice of heart obtained from heart ventricles,
b) placing the slice of step a) on the upper surface of a semiporous support which is permeable to a culture medium,
c) placing the support of step b) onto a culture medium, the slice being fed through the semi-porous support by capillarity.

2. (canceled)

3. A method according to claim 1, wherein the slice culture is obtained from a mammalian heart.

4. A method according to claim 1, wherein the slice culture is obtained from an embryonic mammalian heart.

5. A method according to claim 1, wherein the slice culture is obtainable from a rodent heart.

6. A method according to claim 1, wherein the slice culture is obtained from a primate heart.

7. A method according to claim 1, wherein the thickness of the slice culture is between 800 and 1200 μm.

8. A method according to claim 1, wherein the semiporous support comprises a semiporous membrane sealed to a polystyrene holder.

9. A method according to claim 1, wherein the culture is viable for more than 4 weeks.

10. A method according to claim 1, wherein the culture is viable for more than 2 months.

11-15. (canceled)

16. A system comprising a heart organotypic slice culture on a semiporous support.

17. A system according to claim 16, wherein the slice culture is obtained from a mammalian heart.

18. A system according to claim 16, wherein the slice culture is obtained from an embryonic mammalian heart.

19. A system according to claim 16, wherein the slice culture is obtained from a rodent heart.

20. A system according to claim 16, wherein the slice culture is obtained from a primate heart.

21. A system according to claim 16, wherein the thickness of the slice culture is between 800 and 1200 μm.

22. A system according to claim 16, wherein the semiporous support comprises a semiporous membrane sealed to a polystyrene holder.

23. A system according to claim 16, wherein the culture is viable for more than 4 weeks.

24. A system according to claim 16, wherein the culture is viable for more than 2 months.

25. A method for studying heart physiology comprising the steps of:
a) providing a slice of heart obtained from heart ventricles,
b) placing the slice of step a) on the upper surface of a semiporous support which is permeable to a culture medium,
c) placing the support of step b) onto a culture medium, the slice being fed through the semi-porous support by capillarity,
d) studying the contractions in said slice.

26. A method for screening drugs which comprises the steps of:
a) providing a slice of heart obtained from heart ventricles,
b) placing the slice of step a) on the upper surface of a semiporous support which is permeable to a culture medium,
c) placing the support of step b) onto a culture medium, the slice being fed through the semi-porous support by capillarity,
d) adding a drug to the culture medium,
e) studying the contractions in said slice.

27. A method for studying cell transplantation comprising the steps of:
a) providing a slice of heart obtained from heart ventricles,
b) placing the slice of step a) on the upper surface of a semiporous support which is permeable to a culture medium,
c) placing the support of step b) onto a culture medium, the slice being fed through the semi-porous support by capillarity,
d) transplanting cells into said slice.


SEQUENCE LISTING (4 sequences)

SEQ ID NO: 1 - DNA, 21 bp, Artificial Sequence
  Primer
gaggagaatggccagcaggaa

SEQ ID NO: 2 - DNA, 21 bp, Artificial Sequence
  Primer
gcgaacatctgctccacctca

SEQ ID NO: 3 - DNA, 25 bp, Artificial Sequence
  Primer
ccggacagtgtggcaaccagatcgg

SEQ ID NO: 4 - DNA, 25 bp, Artificial Sequence
  Primer
tggccaaaaggacctgagcgaacgg